SEQUENCE LISTING (11 sequences)

SEQ ID NO: 1 - DNA, 122 bp, Homo sapiens
  gene (1)..(122): CpG region in ADAM12
tgtggaatgatttacaatttacagacatgtgatctcattgagtccattctcccactctctcggagacactatgaggtaagagaaccaccattttgagataataccggagcagccgggaaaac

SEQ ID NO: 2 - DNA, 122 bp, Homo sapiens
  gene (1)..(122): CpG region in NTN4
cccgctggtcccggtcgcctcagcctggcccccggctgccctggcgcccctcctggcccgcgccctgcatctccctagtgacccccctggtgccctgcagcacccacaggcctcccctggga

SEQ ID NO: 3 - DNA, 122 bp, Homo sapiens
  promoter (1)..(122): CpG region in the promoter of PTGS2
tgtgagctccacgggtcaccaatataaagtttcctgccttctgatggacaaaggaagcggcgatggccagaatttgcagggacgctaaatgtccaaaacgtatgccttaaggcatttctctc

SEQ ID NO: 4 - DNA, 20 bp, Artificial Sequence
  human ADAM12 primer (forward)
acaggaagaactgccactgc

SEQ ID NO: 5 - DNA, 20 bp, Artificial Sequence
  human ADAM12 primer (reverse)
ccttggttatctgcttgccg

SEQ ID NO: 6 - DNA, 20 bp, Artificial Sequence
  human NTN4 primer (forward)
ttccgtcccgtgcacaataa

SEQ ID NO: 7 - DNA, 22 bp, Artificial Sequence
  human NTN4 primer (reverse)
acattcgcatttacctgagtgt

SEQ ID NO: 8 - DNA, 19 bp, Artificial Sequence
  human PTGS2 primer (forward)
caaattgctggcagggttg

SEQ ID NO: 9 - DNA, 21 bp, Artificial Sequence
  human PTGS2 primer (reverse)
ctctggtcaatggaagcctgt

SEQ ID NO: 10 - DNA, 20 bp, Artificial Sequence
  human GAPDH primer (forward)
aatcccatcaccatcttcca

SEQ ID NO: 11 - DNA, 20 bp, Artificial Sequence
  human GAPDH primer (reverse)
tggactccacgacgtactca

(19) United States
(12) Patent Application Publication
(10) Pub. No.: US 2016/0265061 A1
(43) Pub. Date: Sep. 15, 2016

(54) COMPOSITION FOR DIAGNOSING OVARIAN CANCER METASTASIS BY USING CPG METHYLATION IN GENE, AND USE THEREOF

(71) Applicant: EWHA UNIVERSITY - INDUSTRY COLLABORATION FOUNDATION, Seoul (KR)

(72) Inventors: Jung-Hyuck AHN, Seoul (KR); Woong JU, Seoul (KR); Hye-Youn SUNG, Seoul (KR)

(73) Assignee: EWHA UNIVERSITY - INDUSTRY COLLABORATION FOUNDATION, Seoul (KR)

(21) Appl. No.: 14/433,397

(22) PCT Filed: Sep. 4, 2014
(86) PCT No.: PCT/KR2014/008325

§ 371 Date: Apr. 3, 2015

(30) Foreign Application Priority Data
Sep. 6, 2013 (KR) 10-2013-0107542
Sep. 6, 2013 (KR) 10-2013-0107543
Sep. 6, 2013 (KR) 10-2013-0107544

(51) Int. Cl. C12Q 1/68

(52) U.S. Cl. CPC C12Q 1/6886; C12Q 2600/154; C12Q 2600/118

(57) ABSTRACT

The present invention relates to a composition, a kit and a method for diagnosing ovarian cancer metastasis or predicting risk of the metastasis by detecting methylation levels at CpG sites of one or more genes selected from the group consisting of ADAM12 (a disintegrin and metalloproteinase 12), NTN4 (netrin 4), and PTGS2 (prostaglandin-endoperoxide synthase 2).

### CROSS REFERENCE TO RELATED APPLICATION

This application claims priority to and the benefit of Korean Patent Application No. 10-2013-0107542 filed on Sep. 6, 2013, Korean Patent Application No. 10-2013-0107543 filed on Sep. 6, 2013, and Korean Patent Application No. 10-2013-0107544 filed on Sep. 6, 2013, which are hereby incorporated by reference for all purposes as if fully set forth herein.

### BACKGROUND OF THE INVENTION

(a) Field of the Invention

The present invention relates to a composition, a kit and a method for diagnosing ovarian cancer metastasis or predicting risk of the metastasis by detecting methylation levels at CpG sites of one or more genes selected from the group consisting of ADAM12 (a disintegrin and metalloproteinase 12), NTN4 (netrin 4), and PTGS2 (prostaglandin-endoperoxide synthase 2).

(b) Description of the Related Art

Ovarian cancer is an intractable cancer having the highest mortality rate of female cancers, and the incidence continues to increase with westernized lifestyle, hormone replacement therapy, and increasing aged populations. There are no distinct symptoms of early ovarian cancer. It has been reported that more than about 70% of patients are diagnosed with advanced ovarian cancer at stage 3 or greater and more than about 75% of patients experience a recurrence or metastasis within the first 2 years after initial treatment.

The treatment for ovarian cancer depends on the type and stage of cancer, which includes surgery, radiation therapy, chemotherapy or the like. These treatment methods show no great therapeutic effects on metastatic cancer from recurrence, because cancer metastasis is accompanied by angiogenesis and cell migration and is a different process from cancer itself. Thus, angiogenesis and cell migration should be also prevented in order to prevent cancer metastasis, because anti-metastatic and anticancer actions are different from each other. Accordingly, diagnosis of cancer is important, but development of biomarkers for predicting recurrence and metastasis after treatment of ovarian cancer is expected to greatly contribute to improvement of survival rate and treatment efficiency. Further, prediction of cancer recurrence and metastasis requires development of biomarkers that are different from the cancer diagnostic biomarkers, because there is an underlying difference between cancer metastasis or recurrence and tumorigenesis.

In more detail, it has been reported that metastatic cancer has biological characteristics different from those of the primary cancer, because there are differences in gene expression patterns between metastatic cancer and primary cancer. For instance, various growth hormones are needed for tumor cell growth, and changes in gene expression favorable to survival of metastatic cancer cells are ultimately required because metastatic cancer cells must overcome the anticancer effects to survive. It seems that these expression patterns play a very important role in determining the cancer metastasis. Therefore, it is hard to impute a cause of metastasis to a high expression level of a single gene of the related genes in tumor cells (primary site).

On the other hand, Korean Patent NO. 1169127, Japanese Patent Publication NO. 2010-178650, and US Patent Publication NO. 2010-0279301 disclose that the genes selected in the present invention can be used as diagnostic markers for various cancers. However, the present invention clearly differs from these documents in that recurrence and metastasis of ovarian cancer are diagnosed or predicted using site-specific hypermethylation at specific CpG sites of the corresponding genes.

### SUMMARY OF THE INVENTION

In the present invention, gene expression patterns between primary cancer cells and metastatic tissues were compared. Of the genes showing changes in their expression patterns in the metastatic tissues, genes, of which CpG methylation changes are found to affect gene expressions, were finally selected. Furthermore, the specific CpG sites affecting the gene expressions were identified, and methylation levels at the specific CpG sites of the corresponding genes were measured so as to predict the risk of ovarian cancer metastasis, leading to the present invention.

An object of the present invention is to provide a composition for diagnosing ovarian cancer metastasis or predicting risk of the metastasis, including an agent measuring methylation levels at the CpG sites of one or more genes selected from the group consisting of ADAM12 (a disintegrin and metalloproteinase 12), NTN4 (netrin 4), and PTGS2 (prostaglandin-endoperoxide synthase 2).

Another object of the present invention is to provide a kit for diagnosing ovarian cancer metastasis or predicting risk of the metastasis, including the composition.

Still another object of the present invention is to provide a method for diagnosing ovarian cancer metastasis or predicting risk of the metastasis by measuring methylation levels at the CpG sites of the genes.

Still another object of the present invention is to provide a method for providing information for diagnosing ovarian cancer metastasis or predicting risk of the metastasis, including the step of measuring the methylation levels at the CpG sites of the genes in a biological sample obtained from a patient suspected of having ovarian cancer metastasis.

Still another object of the present invention is to provide a method for diagnosing ovarian cancer metastasis or risk of the metastasis, including the steps of:

(a) measuring methylation levels at the CpG sites of one or more genes selected from the group consisting of ADAM12, NTN4 and PTGS2 in a biological sample of a subject,

(b) comparing the methylation levels with those of the genes of a control sample, and

(c) determining that the subject has ovarian cancer metastasis or is at the risk of the metastasis, when the methylation levels measured in the sample of the subject are higher than those of the control sample.

### Effect of the Invention

According to the present invention, methylation levels at CpG sites of specific genes of genomic DNA which is collected from a biological sample of a patient may be measured by a PCR-based MSP method (methylation-specific PCR) so as to diagnose the risk of ovarian cancer metastasis within several hours, thereby developing a convenient diagnostic kit with high accuracy.

### BRIEF DESCRIPTION OF THE DRAWINGS

FIG. 1 is a diagram showing integration of mRNA and CpG methylation data;

FIG. 2 is a photograph showing construction of an ovarian cancer metastasis animal model by injecting SK-OV-3 cell line into the intraperitoneal cavity of a nude mouse;

FIG. 3 is the result of showing the distribution patterns of global DNA methylation in primary ovarian cancer cell line (SK-OV-3) and tumor tissues of 7 animals with ovarian cancer metastasis (n=7; designated as 1C˜8C);

FIG. 4 is the Heatmap result of the genes showing significant expression changes in the metastatic tumor tissues, compared to the primary ovarian cancer cell line;

FIG. 5 is the result of showing changes in the DNA methylation and gene expression in ovarian cancer metastasis animal model;

FIG. 6 is the result of qRT-PCR showing changes in ADAM12 gene expression in the tumor tissues of ovarian cancer metastasis animal models (n=7; designated as 1C˜8C);

FIG. 7 is the result of qRT-PCR showing changes in NTN4 gene expression in the tumor tissues of ovarian cancer metastasis animal models (n=7; designated as 1C˜8C);

FIG. 8 is the result of qRT-PCR showing changes in PTGS2 gene expression in the tumor tissues of ovarian cancer metastasis animal models (n=7; designated as 1C˜8C);

FIG. 9 is the result of DNA methylation microarray for analyzing DNA methylation at the CpG site of ADAM12 gene in the tumor tissues of ovarian cancer metastasis animal models (n=7; designated as 1C˜8C);

FIG. 10 is the result of DNA methylation microarray for analyzing DNA methylation at the CpG site of NTN4 gene in the tumor tissues of ovarian cancer metastasis animal models (n=7; designated as 1C˜8C);

FIG. 11 is the result of DNA methylation microarray for analyzing DNA methylation at the CpG site of PTGS2 gene in the tumor tissues of ovarian cancer metastasis animal models (n=7; designated as 1C˜8C);

FIG. 12 is the result of analyzing changes in ADAM12 gene expression after treatment of SK-OV-3 cell line with 5-aza-2′-deoxycytidin;

FIG. 13 is the result of analyzing changes in NTN4 gene expression after treatment of SK-OV-3 cell line with 5-aza-2′-deoxycytidin; and

FIG. 14 is the result of analyzing changes in PTGS2 gene expression after treatment of SK-OV-3 cell line with 5-aza-2′-deoxycytidin.

### DETAILED DESCRIPTION OF THE EMBODIMENTS

Based on the finding that the specific CpG sites of ADAM12, NTN4 and PTGS2 genes are specifically hypermethylated in metastatic ovarian cancer tissues, the present invention provides a technique of diagnosing ovarian cancer metastasis or predicting risk of the metastasis by using the methylation levels of these genes as biomarkers.

Since the CpG sites of ADAM12, NTN4 and PTGS2 genes are specifically hypermethylated in metastatic ovarian cancer tissues, respectively, each of them can be used as a single biomarker for diagnosing ovarian cancer metastasis or predicting risk of the metastasis, or two or more thereof can be used as multi-biomarkers.

Accordingly, in an aspect, the present invention relates to a composition for diagnosing ovarian cancer metastasis or predicting risk of the metastasis including an agent measuring methylation levels at the CpG sites of one or more genes selected from the group consisting of ADAM12, NTN4 and PTGS2, and a kit including the same.

In a preferred embodiment, the present invention relates to a composition for diagnosing ovarian cancer metastasis or predicting risk of the metastasis including an agent measuring the methylation level at the CpG site of ADAM12 gene, and a kit including the same.

In this case, more preferably, the composition and the kit may further include an agent measuring methylation levels at the CpG sites of one or more genes selected from the group consisting of NTN4 and PTGS2.

In another preferred embodiment, the present invention relates to a composition for diagnosing ovarian cancer metastasis or predicting risk of the metastasis including an agent measuring the methylation level at the CpG site of NTN4 gene, and a kit including the same.

In this case, more preferably, the composition and the kit may further include an agent measuring methylation levels at the CpG sites of one or more genes selected from the group consisting of ADAM12 and PTGS2.

In another preferred embodiment, the present invention relates to a composition for diagnosing ovarian cancer metastasis or predicting risk of the metastasis including an agent measuring the methylation level at the CpG site of PTGS2 gene, and a kit including the same.

In this case, more preferably, the composition and the kit may further include an agent measuring methylation levels at the CpG sites of one or more genes selected from the group consisting of ADAM12 and NTN4.

In the present invention, the sequence information of mRNAs of ADAM12, NTN4 and PTGS2 genes may be obtained from the known gene database. For example, the nucleotide sequence of human ADAM12 gene may be obtained from GenBank Accession NO. NM_003474, the nucleotide sequence of human NTN4 gene may be obtained from GenBank Accession NO. NM_021229, and the nucleotide sequence of human PTGS2 gene may be obtained from GenBank Accession NO. NM_000963.

As used herein, the term “methylation” refers to attachment of methyl groups to bases constituting DNA. Preferably, the methylation, as used herein, means methylation that occurs at cytosines of specific CpG sites in a particular gene. If methylation occurs, binding of transcription factors is inhibited to suppress expression of a particular gene. If non-methylation or hypomethylation occurs, expression of the particular gene is increased.

In the genomic DNA of mammalian cells, there is the fifth base in addition to A, C, G and T, namely, 5-methylcytosine (5-mC), in which a methyl group is attached to the fifth carbon of the cytosine ring. Methylation of 5-methylcytosine is always attached only to the C of a CG dinucleotide (5′-mCG-3′), which is frequently marked CpG. The methylation of this CpG inhibits a repetitive sequence in genomes, such as alu or transposon, from being expressed. Also, 5-mC of this CpG is naturally deaminated to thymine (T), and thus CpG is a site where an epigenetic change in mammalian cells appears most often.

As used herein, the phrase “measuring the methylation level” means determination of the methylation levels at CpG sites of ADAM12, NTN4 and/or PTGS2 gene(s), and the methylation level may be determined by, for example, methylation-specific PCR (methylation-specific polymerase chain reaction, MSP), real time methylation-specific PCR (real time methylation-specific polymerase chain reaction), PCR using a methylation DNA-specific binding protein, and quantitative PCR. Alternatively, it may be determined by automatic sequencing such as pyrosequencing and bisulfite sequencing, but is not limited thereto.

Preferably, the CpG sites of ADAM12, NTN4 and/or PTGS2 gene(s) mean CpG sites that exist on DNAs of the genes. The DNA of the gene is a concept encompassing a series of components that are needed for gene expression and operably linked to each other, and for example, includes a promoter region, a protein coding region (open reading frame, ORF) and a terminator region. Therefore, the CpG sites of ADAM12, NTN4 and/or PTGS2 gene(s) may exist in the promoter region, the protein coding region (open reading frame, ORF), or the terminator region.

Preferably, measurement of the methylation level at the CpG site of the ADAM12 gene in the present invention may mean measurement of the methylation level of cytosine at the CpG site at position from 127779782 to 127779903 of chromosome 10. In the present invention, the base sequence at position from 127779782 to 127779903 of chromosome 10 is represented by SEQ ID NO. 1.

More preferably, measurement of the methylation level at the CpG site of the ADAM12 gene in the present invention may mean measurement of the methylation level of cytosine at the position 127779842 (at position 61 of SEQ ID NO. 1) of chromosome 10.

Preferably, measurement of the methylation level at the CpG site of the NTN4 gene in the present invention may mean measurement of the methylation level of cytosine at the CpG site at position from 96184755 to 96184876 of chromosome 12. In the present invention, the base sequence at position from 96184755 to 96184876 of chromosome 12 is represented by SEQ ID NO. 2.

More preferably, measurement of the methylation level at the CpG site of the NTN4 gene in the present invention may mean measurement of the methylation level of cytosine at position 96184815 (at position 61 of SEQ ID NO. 2) of chromosome 12.

Preferably, measurement of the methylation level at the CpG site of the PTGS2 gene in the present invention may mean measurement of the methylation level of cytosine at the CpG site at position from 186650381 to 186650502 of chromosome 1. In the present invention, the base sequence at position from 186650381 to 186650502 of chromosome 1 is represented by SEQ ID NO. 3.

More preferably, measurement of the methylation level at the CpG site of the PTGS2 gene in the present invention may mean measurement of the methylation level of cytosine at position 186650441 (at position 61 of SEQ ID NO. 3) of chromosome 1.

In the present invention, the base sequences of the human genomic chromosomes are given according to the latest February 2009 Human reference sequence (GRCh 37), but the specific sequences of the human genomic chromosomes may be slightly revised according to update of the genomic sequence analysis. The annotation of the human genomic locations of the present invention may differ depending on the revision. Therefore, although the annotation of the human genomic locations according to the February 2009 Human reference sequence (GRCh37) is revised according to the human reference sequence updated after the filing date of the present application, it will be apparent that the revised annotation of human genomic locations is also within the scope of the present invention. Such revision may be readily apparent to those skilled in the art to which the present invention pertains.

Based on the finding that there are differences in gene expressions between primary tumors at the early stage and metastatic tumors, the present inventors compared the gene expression patterns between primary cancer cell lines and metastatic tissues to finally select genes, in which changes in CpG methylation were found to affect gene expressions, from the genes showing gene expression changes in metastatic tissues. Furthermore, they identified the specific CpG sites that affect the gene expressions, and also found that risk of the metastasis may be predicted by measuring methylation levels at the specific CpG sites of the corresponding genes.

In more detail, the present inventors constructed ovarian cancer metastasis animal model by injecting the primary ovarian cancer cell line SK-OV-3 into the intraperitoneal cavity of 10 nude mice, and they extracted genomic DNAs and RNAs from the tumor tissues of these animal models to carry out DNA methylation microarray using an Illumina Human Methylation 450 BeadChip and gene expression microarray using an Affymetrix Human Gene 1.0 ST. Through the integration analysis of the results, they selected genes, in which changes in CpG methylation were suspected to affect gene expressions.

Of the selected genes, the ovarian cancer metastasis mouse model showed up to 11-19-fold decrease in ADAM12 expression, and about 2.2-2.5-fold increase in DNA methylation, compared to the primary cancer cell line. The ovarian cancer metastasis mouse model showed up to 1.8-6.9-fold decrease in NTN4 expression, and about 2.7-4.0-fold increase in DNA methylation at the specific CpG site, compared to the primary cancer cell line. The ovarian cancer metastasis mouse model showed up to 1.5-17.4-fold decrease in PTGS2 expression, and about 1.6-fold increase in DNA methylation at the specific CpG site, compared to the primary cancer cell line.

Further, treatment of the primary cell line SKOV-3 with a DNA demethylating agent, 5-aza-2′-deoxycytidine resulted in about 2-fold increase in ADAM12, NTN4 and PTGS2 gene expressions, respectively indicating that expressions of the above genes are regulated by DNA methylation.

Therefore, hypermethylation of DNA methylation at the specific CpG site of ADAM12, NTN4 and/or PTGS2 may be utilized as biomarkers for diagnosing ovarian cancer metastasis or predicting risk of the metastasis.

As used herein, the term “diagnosis of metastasis” means examination of ovarian cancer metastasized to other tissues from the ovary. In general, ovarian cancer spreads to other organ tissues through the peritoneal cavity. The tissues other than the ovary may be, for example, various organ tissues within the peritoneal cavity including the large intestine, small intestine, and periphery of the liver. More preferably, diagnosis of metastasis, as used herein, means examination of metastatic status of ovarian cancer by distinguishing a sample of a patient with metastasis from the non-metastatic, primary ovarian cancer sample.

As used herein, the term “prediction of risk of the metastasis” means prediction of spreading possibility of ovarian cancer from the ovary to other tissues. More preferably, the prediction of risk of the metastasis, as used herein, means prediction of possibility of recurrence and metastasis of ovarian cancer in the treated tissue after treating a patient having metastatic ovarian cancer with therapy such as surgery, radiation therapy, chemotherapy or the like. From another point of view, the prediction of risk of the metastasis, as used herein, means prediction of possibility of metastasis in a patient with ovarian cancer by distinguishing a sample of the patient at the risk of the metastasis from the non-metastatic, primary ovarian cancer sample.

Further, aberrant methylation in cancer tissues is considerably similar to methylation of genomic DNA obtained from a biological sample such as cells, whole blood, serum, plasma, saliva, sputum, cerebrospinal fluid or urine. Therefore, when the markers of the present invention are used, there is an advantage that it is possible to diagnose ovarian cancer metastasis or to predict risk of the metastasis in the blood or body fluid in a simple manner.

In the present invention, the agent measuring a methylation level at the CpG site may include a compound modifying an unmethylated cytosine base or a methylation-sensitive restriction enzyme, primers specific to the methylated allele sequences of ADAM12, NTN4 and/or PTGS2 gene(s), and primers specific to the unmethylated allele sequence of the gene.

The compound modifying an unmethylated cytosine base may be bisulfite or a salt thereof, but is not limited thereto, preferably sodium bisulfite. A method of detecting methylation at the CpG site by modifying the unmethylated cytosine residue using bisulfite is widely known in the art (WO01/26536; US2003/0148326A1).

Further, the methylation-sensitive restriction enzyme is a restriction enzyme capable of specifically detecting CpG methylation, and preferably a restriction enzyme including CG as a restriction enzyme recognition site. Examples thereof include SmaI, SacII, EagI, HpaII, MspI, BssHII, BstUI, NotI or the like, but are not limited thereto. Cleavage by a restriction enzyme differs depending on methylation or unmethylation of C at the restriction enzyme recognition site, and the methylation may be detected by PCR or Southern blot analysis. In addition to the restriction enzymes, other methylation-sensitive restriction enzymes are well known in the art.

The methylation levels of the particular CpG sites of ADAM12, NTN4 and PTGS2 genes in an individual suspected of having ovarian cancer metastasis may be determined by obtaining genomic DNA from a biological sample of the individual, treating the obtained DNA with a compound modifying an unmethylated cytosine base or a methylation-sensitive restriction enzyme, amplifying the treated DNA using primers by PCR, and then identifying the presence of the resulting amplified product.

Therefore, the agent of the present invention may include primers specific to the methylated allele sequences of ADAM12, NTN4 and PTGS2 genes, and primers specific to the unmethylated allele sequences of the genes. As used herein, the term “primer” means a short nucleic acid sequence having a free 3′ hydroxyl group, which is able to form base-pairing with a complementary template and serves as a starting point for replication of the template strand. A primer is able to initiate DNA synthesis in the presence of a reagent for polymerization (i.e., DNA polymerase or reverse transcriptase) and four different nucleoside triphosphates at suitable buffers and temperature. In addition, the primers are sense and antisense nucleic acids having a sequence of 7 to 50 nucleotides. The primer may have additional properties that do not change the nature of the primer to serve as a starting point for DNA synthesis.

The primers of the present invention can be designed according to the particular CpG sequence that is subjected to methylation analysis, and may be a set of primers that are able to specifically amplify bisulfite-unmodified cytosine due to methylation and a set of primers that are able to specifically amplify bisulfite-modified cytosine due to unmethylation.

The composition and kit may further include polymerase, agarose, and a buffer solution needed for electrophoresis, in addition to the above agent.

In another aspect, the present invention relates to a method for diagnosing ovarian cancer metastasis or predicting risk of the metastasis by measuring methylation levels at the CpG sites of one or more genes selected from the group consisting of ADAM12, NTN4 and PTGS2.

For example, the present invention relates to a method for diagnosing ovarian cancer metastasis or risk of the metastasis, including the steps of:

(a) measuring methylation levels at the CpG sites of one or more genes selected from the group consisting of ADAM12, NTN4 and PTGS2 in a biological sample of a subject,

(b) comparing the methylation levels with those of the genes of a control sample, and

(c) determining that the subject has ovarian cancer metastasis or is at the risk of the metastasis, when the methylation levels measured in the sample of the subject are higher than those of the control sample.

Preferably, the control sample may be a sample of a subject with non-metastatic ovarian cancer, or a control sample of primary ovarian cancer.

In a preferred embodiment, the present invention relates to a method for diagnosing ovarian cancer metastasis or risk of the metastasis, including the steps of:

measuring methylation level at the CpG site of ADAM12 gene in a biological sample of a subject,

comparing the methylation level with that of the gene of a control sample, and

determining that the subject has ovarian cancer metastasis or is at the risk of the metastasis, when the methylation level measured in the sample of the subject is higher than that of the control sample.

In this case, more preferably, the method may further include the steps of:

measuring methylation levels at the CpG sites of one or more genes selected from the group consisting of NTN4 and PTGS2 in the biological sample of the subject,

comparing the methylation levels with those of the genes of the control sample, and

determining that the subject has ovarian cancer metastasis or is at the risk of the metastasis, when the methylation levels measured in the sample of the subject are higher than those of the control sample.

In another preferred embodiment, the present invention relates to a method for diagnosing ovarian cancer metastasis or risk of the metastasis, including the steps of:

measuring methylation level at the CpG site of NTN4 gene in a biological sample of a subject,

comparing the methylation level with that of the gene of a control sample, and

determining that the subject has ovarian cancer metastasis or is at the risk of the metastasis, when the methylation level measured in the sample of the subject is higher than that of the control sample.

In this case, more preferably, the method may further include the steps of:

measuring methylation levels at the CpG sites of one or more genes selected from the group consisting of ADAM12 and PTGS2 in the biological sample of the subject,

comparing the methylation levels with those of the genes of the control sample, and

determining that the subject has ovarian cancer metastasis or is at the risk of the metastasis, when the methylation levels measured in the sample of the subject are higher than those of the control sample.

In another preferred embodiment, the present invention relates to a method for diagnosing ovarian cancer metastasis or risk of the metastasis, including the steps of:

measuring methylation level at the CpG site of PTGS2 gene in a biological sample of a subject,

comparing the methylation level with that of the gene of a control sample, and

determining that the subject has ovarian cancer metastasis or is at the risk of the metastasis, when the methylation level measured in the sample of the subject is higher than that of the control sample.

In this case, more preferably, the method may further include the steps of:

measuring methylation levels at the CpG sites of one or more genes selected from the group consisting of ADAM12 and NTN4 in the biological sample of the subject,

comparing the methylation levels with those of the genes of the control sample, and

determining that the subject has ovarian cancer metastasis or is at the risk of the metastasis, when the methylation levels measured in the sample of the subject are higher than those of the control sample.

As used herein, the term “biological sample” includes samples displaying a difference in the methylation levels of ADAM12, NTN4 and/or PTGS2 gene(s) due to the ovarian cancer metastasis, such as tissues, cells, whole blood, serum, plasma, saliva, sputum, or urine, but is not limited thereto.

First, to measure the methylation level of genomic DNAs obtained from patients suspected of having ovarian cancer metastasis, the genomic DNAs may be obtained by a phenol/chloroform extraction method, an SDS extraction method (Tai et al., Plant Mol. Biol. Reporter, 8: 297-303, 1990), or a CTAB separation method (Cetyl Trimethyl Ammonium Bromide; Murray et al., Nuc. Res., 4321-4325, 1980) typically used in the art, or using a commercially available DNA extraction kit.

The step of (a) measuring methylation levels at the CpG sites of genes may be performed by using a compound modifying an unmethylated cytosine base or a methylation sensitive restriction enzyme, primers specific to the methylated sequence at the CpG site of the gene, and primers specific to the unmethylated sequence.

In more detail, the step may be performed by a step of treating the genomic DNA obtained from the sample with the compound modifying an unmethylated cytosine base or the methylation sensitive restriction enzyme; and

a step of measuring the methylation level of the treated DNA by one or more methods selected from the group consisting of methylation-specific polymerase chain reaction, real time methylation-specific polymerase chain reaction, PCR using a methylated DNA-specific binding protein, quantitative PCR, pyrosequencing and bisulfite sequencing using primers capable of amplifying the methylated region at CpG site of the gene.

In the above, the compound modifying unmethylated cytosine base may be bisulfite, and preferably sodium bisulfite. The method of detecting gene methylation by modifying unmethylated cytosine residues using bisulfite is widely known in the art.

Further, the methylation-sensitive restriction enzyme is, as described above, a restriction enzyme capable of specifically detecting methylation of the particular CpG site, and preferably a restriction enzyme containing CG as a restriction enzyme recognition site. Examples thereof include SmaI, SacII, EagI, HpaII, MspI, BssHII, BstUI, NotI or the like, but are not limited thereto.

The primers used herein are, as described above, designed according to the particular CpG site that is subjected to methylation analysis, and may be a set of primers that are able to specifically amplify bisulfite-unmodified cytosine due to methylation and a set of primers that are able to specifically amplify bisulfite-modified cytosine due to unmethylation.

Measurement of the methylation level may be conducted by a method known in the art. For example, electrophoresis is performed to detect the presence of a band at the desired size. For example, in the case of using the compound modifying the unmethylated cytosine residues, methylation may be determined according to the presence of the PCR product that is amplified by the two types of primer pairs, that is, a set of primers that are able to specifically amplify bisulfite-unmodified cytosine due to methylation and a set of primers that are able to specifically amplify bisulfite-modified cytosine due to unmethylation. Preferably, methylation may be determined by treating genomic DNA of a sample with bisulfite, amplifying the CpG site of the corresponding gene by PCR, and then analyzing the amplified base sequence by a bisulfite genomic sequencing method.

Further, if a restriction enzyme is used, methylation may be determined by a method known in the art. For example, when the PCR product is present in the restriction enzyme-treated DNA, under the state where the PCR product is present in the mock DNA, it is determined as gene methylation. When the PCR product is absent in the restriction enzyme-treated DNA, it is determined as gene unmethylation. In this way, the methylation can be determined, which is apparent to those skilled in the art. The term ‘mock DNA’ refers to a sample DNA that is isolated from a sample and then undergoes no treatment.

When hypermethylation at the CpG site of ADAM12, NTN4 and/or PTGS2 gene(s) is observed in a sample of a patient by the above method, it may be predicted that the patient has ovarian cancer metastasis or is at the risk of the metastasis.

Therefore, the method of the present invention is used to effectively examine the CpG methylation of ADAM12, NTN4 and/or PTGS2 gene(s), thereby diagnosing ovarian cancer metastasis or predicting risk of the metastasis.

### DETAILED DESCRIPTION OF THE EMBODIMENTS

Hereinafter, the present invention will be described in more detail with reference to Examples. However, the following Examples are for illustrative purposes only, and the present invention is not intended to be limited thereto.

### Example 1

### Cell Line and Ovarian Cancer Metastasis Mouse Model

Human ovarian cancer cell line SK-OV-3 was purchased from American type culture collection (ATCC no. HTB-77) and cultured in a McCoy's 5a medium containing 10% FBS (fetal bovine serum), 100 U/mL of penicillin and 100 μg/mL of streptomycin.

In order to prepare ovarian cancer metastasis mouse model, 2×10⁶ SK-OV-3 cells were suspended in the cell culture medium, and injected into the peritoneal cavity of 10 4-6-week old female BALB/c nude mice. 4 weeks later, tumor tissues (organ tissues in the peritoneal cavity, including the large intestine, small intestine, and periphery of the liver) formed by migration of the cell line along the peritoneal cavity were excised and stored in liquid nitrogen.

### Example 2

### Total RNA Extraction

Total RNAs were extracted from SK-OV-3 cell line and the tumor tissues using an RNeasy mini kit (Qiagen), respectively. The extraction was performed according to manufacturer's instructions. The extracted total RNAs were quantified using a spectrophotometer, and RNA degradation was examined by electrophoresis in a 1% agarose gel.

### Example 3

### Quantitative Real-Time PCR (qRT-PCR)

For cDNA synthesis, Superscript II reverse transcriptase (Invitrogen) was used. 1 μg of total RNA and 50 ng of oligo dT were denatured at 70° C. for 10 minutes, and then mixed with a reaction mixture containing 4 μl of 5×RT buffer, 2 μl of 0.1 mM DTT, 4 μl of 2.5 mM dNTP mixture, 200 units of Superscript II reverse transcriptase and 10 units of RNase inhibitor to prepare 20 μl of a resulting reaction mixture, which was reacted at 25° C. for 10 minutes, at 42° C. for 50 minutes, and at 95° C. for 5 minutes to synthesize cDNA. This cDNA was diluted at 1:4, and 2 μl thereof was used as a template for qRT-PCR. In qRT-PCR, 20 μl of a reaction mixture containing 2 μl of cDNA, 10 μl of SYBR Premix EX Taq (Takara Bio), 0.4 μl of Rox reference dye (50×, Takara Bio), and 200 nM of primers of each gene was reacted at 95° C. for 30 seconds, and then repeated for 40 cycles (at 95° C. for 3 seconds, and at 60° C. for 30 seconds) using an ABI 7500fast sequence detection system (Applied Biosystems) for amplification. The PCR products were reacted at 95° C. for 15 seconds, at 60° C. for 1 minute, and at 95° C. for 15 seconds to examine their specificity. GAPDH expression was used as an internal control, and expressions of ADAM12, NTN4 and PTGS2 genes were normalized using the GAPDH expression level by a ΔΔC_{T} method. The sequences of the primers used are as follows.

TABLE 1

| | | SEQ |
|---|---|---|
| | Sequence | ID NO. |
| human ADAM12 | 5′-ACAGGAAGAACTGCCACTGC-3′ | 4 |
| (forward) | | |
| human ADAM12 | 5′-CCTTGGTTATCTGCTTGCCG-3′ | 5 |
| (reverse) | | |
| human NTN4 | 5′-TTCCGTCCCGTGCACAATAA-3′ | 6 |
| (forward) | | |
| human NTN4 | 5′-ACATTCGCATTTACCTGAGTGT-3′ | 7 |
| (reverse) | | |
| human PTGS2 | 5′-CAAATTGCTGGCAGGGTTG-3′ | 8 |
| (forward) | | |
| human PTGS2 | 5′-CTCTGGTCAATGGAAGCCTGT-3′ | 9 |
| (reverse) | | |
| human GAPDH | 5′-AATCCCATCACCATCTTCCA-3′ | 10 |
| (forward) | | |
| human GAPDH | 5′-TGGACTCCACGACGTACTCA-3′ | 11 |
| (reverse) | | |

### Example 4

### 5-aza-2′-deoxycytidine (5-aza-dC) Treatment

SK-OV-3 cell line was treated with a methylation inhibitor, 5-aza-2′-deoxycytidine (Sigma-Aldrich) at concentrations of 5, 10, and 20 μM for 3 days, and then changes in ADAM12, NTN4 and PTGS2 gene expressions were measured by qRT-PCP.

### Example 5

### mRNA Microarray

mRNA microarray was performed using a GeneChip Human Gene 1.0 ST arrays.

Gene expression values obtained after scanning were subjected to background correction, RMA normalization (Biostatistics. 2003 April; 4(2):249-64. Exploration, normalization, and summaries of high density oligonucleotide array probe level data), and log₂ transformation, and finally used for statistical analysis. In order to identify differentially expressed genes (DEGs) in two groups, a Bayesian t-test (Limma: Linear Models for. Microarray Data. Gordon K. Smyth.) method was used. Finally, genes with p value<0.05 and absolute value of log₂ (fold change) greater than 0.585 were selected as DEG.

### Example 6

### DNA Methylation Microarray

DNA methylation microarray was performed using an Infinium® Human Methylation 450K BeadChip. The level of DNA methylation was reported as a β-value ranging from 0 to 1, with 0 being completely unmethylated and 1 being completely methylated at the corresponding CpG site.

In order to identify differentially methylated genes (DMGs) in two groups, the Bayesian t-test was used. Finally, the CpG sites with p value<0.05 and absolute β-value difference≧0.3 were selected as differentially methylated CpG sites, and of them, genes showing methylation changes at the CpG sites were selected as DMG.

### Example 7

### Integration of DEG and DMG Data

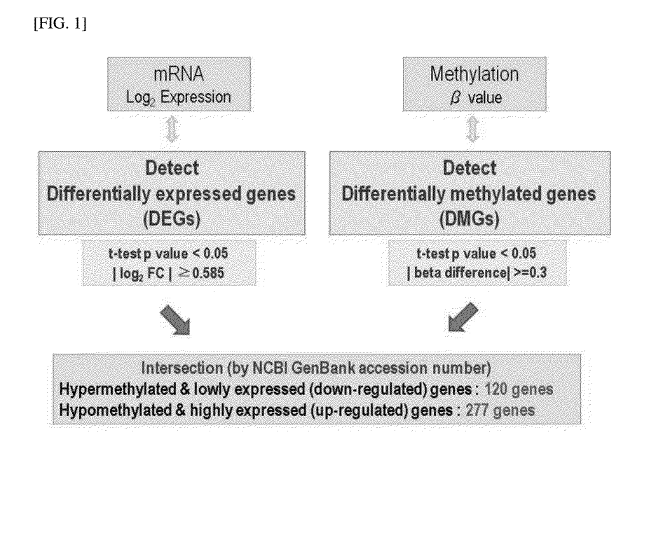

According to the procedure of FIG. 1, DEG and DMG data thus determined were integrated.

Experimental Results

1. Construction of Ovarian Cancer Metastasis Animal Model

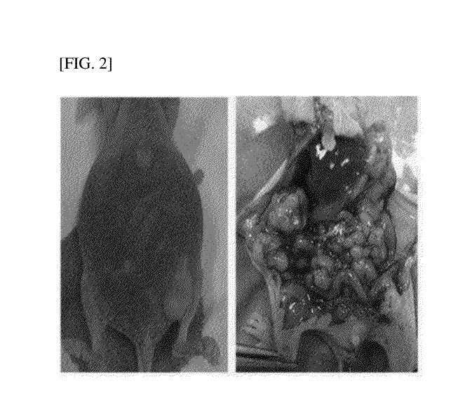

Ovarian cancer metastasis animal models were constructed by injecting the ovarian cancer cell line SK-OV-3 into the intraperitoneal cavity of 10 female nude mice (FIG. 2).

2. Analysis of Epigenetic Change in Ovarian Cancer Metastasis Animal Model

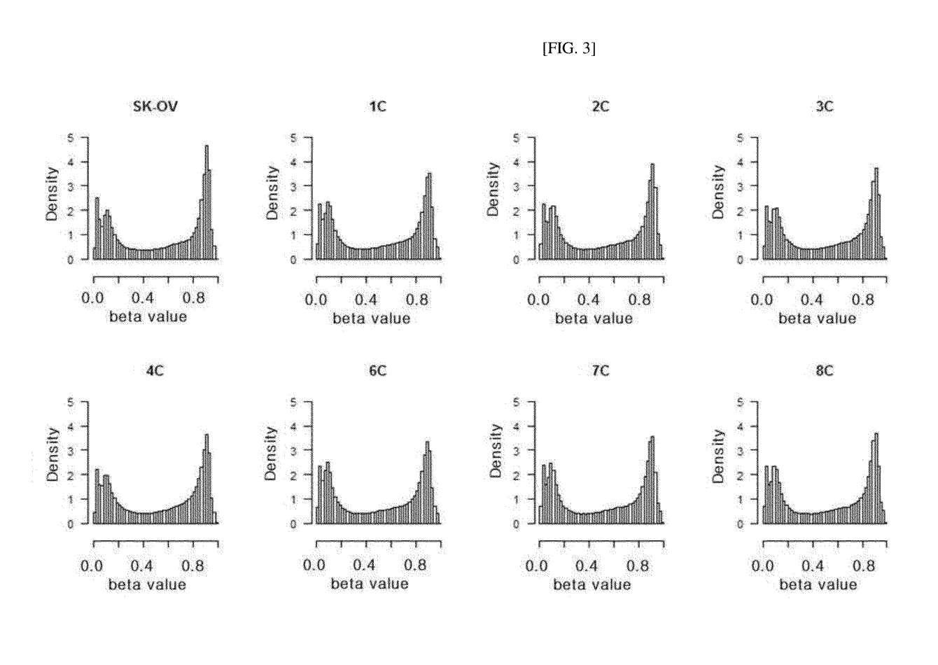

Genomic DNAs were extracted from the tumor tissues (organ tissues in the peritoneal cavity, including the large intestine, small intestine, and periphery of the liver) obtained from metastasis animal model and the ovarian cancer cell line SK-OV-3, and subjected to DNA methylation microarray using an Illumina Human Methylation 450 BeadChip, thereby analyzing CpG sites showing significant changes in DNA methylation in metastatic tumor tissues, compared to the primary ovarian cancer cell line. As a result, decreased global DNA methylation (global hypomethylation) was observed in the metastatic tumor tissues, compared to the primary ovarian cancer cell line (FIG. 3).

3. Analysis of Gene Expression Changes in Ovarian Cancer Metastasis Animal Model

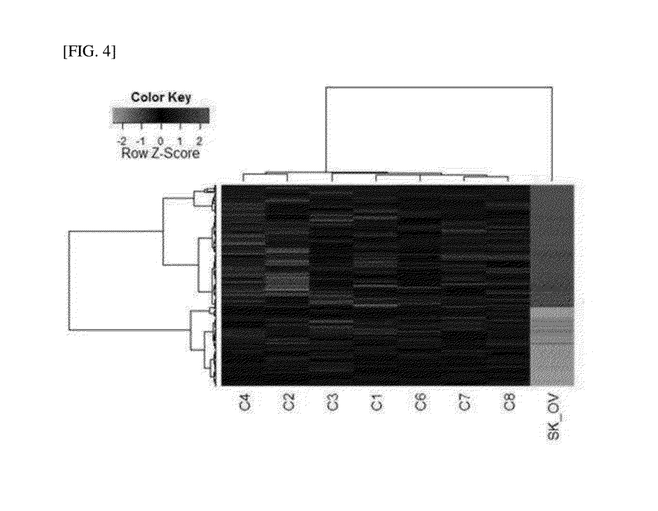

RNAs were extracted from the tumor tissues obtained from metastasis animal model and the ovarian cancer cell line SK-OV-3, and subjected to expression microarray using an Affymetrix Human Gene 1.0 ST, thereby analyzing genes showing significant changes in their expression in metastatic tumor tissues, compared to the primary ovarian cancer cell line (FIG. 4). As a result, expressions of the genes related to cell adhesion, cell cycle, wound healing, and coagulation were increased, whereas expressions of the genes related to transcription, transcriptional regulation, cell death and cell death regulation were remarkably decreased (Table 2).

TABLE 2

| | | Enrichment | Gene function | | | |
|---|---|---|---|---|---|---|
| | Cluster No. | Score | (GOTERM_BP_FAT) | Number | P value | BH p value |
| Increased | Cluster 1 | 8.2 | Cell adhesion | 85 | 2.35E−10 | 1.10E−07 |
| expression | | | Biological adhesion | 85 | 2.52E−10 | 1.01E−07 |
| | Cluster 2 | 7.6 | M phase | 51 | 5.34E−10 | 1.88E−07 |
| | | | Cell cycle | 58 | 1.53E−09 | 4.77E−07 |
| | Cluster 3 | 6.6 | Nucleosome assembly | 27 | 9.48E−13 | 2.67E−09 |
| | | | Chromatin assembly | 27 | 2.36E−12 | 3.31E−09 |
| | Cluster 4 | 5.4 | Calcium-dependent | 11 | 2.70E−07 | 4.22E−05 |
| | | | cell-cell adhesion | | | |
| | | | Extracellular structure | 28 | 9.53E−07 | 1.34E−04 |
| | Cluster 5 | 2.7 | Wound healing | 25 | 3.62E−04 | 0.029 |
| | | | Coagulation | 16 | 8.98E−04 | 0.063 |
| Decreased | Cluster 1 | 5.6 | Transcription | 263 | 2.78E−08 | 1.04E−04 |
| expression | | | Transcriptional | 311 | 1.05E−07 | 1.97E−04 |
| | | | regulation | | | |
| | Cluster 2 | 3.2 | Mitochondria organelle | 29 | 4.72E−05 | 0.029 |
| | | | Protein localization in | 28 | 3.22E−04 | 0.11 |
| | | | cell organelles | | | |
| | Cluster 3 | 3.1 | tRNA metabolism | 27 | 1.96E−05 | 0.018 |
| | | | tRNA aminoacylation | 12 | 0.0025 | 0.27 |
| | Cluster 4 | 3.1 | tRNA metabolism | 27 | 1.96E−05 | 0.018 |
| | | | ncRNA metabolism | 42 | 2.58E−05 | 0.019 |
| | Cluster 5 | 2.5 | Apoptosis regulation | 104 | 3.81E−04 | 0.12 |
| | | | Cell death regulation | 104 | 4.51E−04 | 0.13 |

4. Integrated Analysis of Epigenetic Change and Gene Expression of Ovarian Cancer Metastasis Animal Model

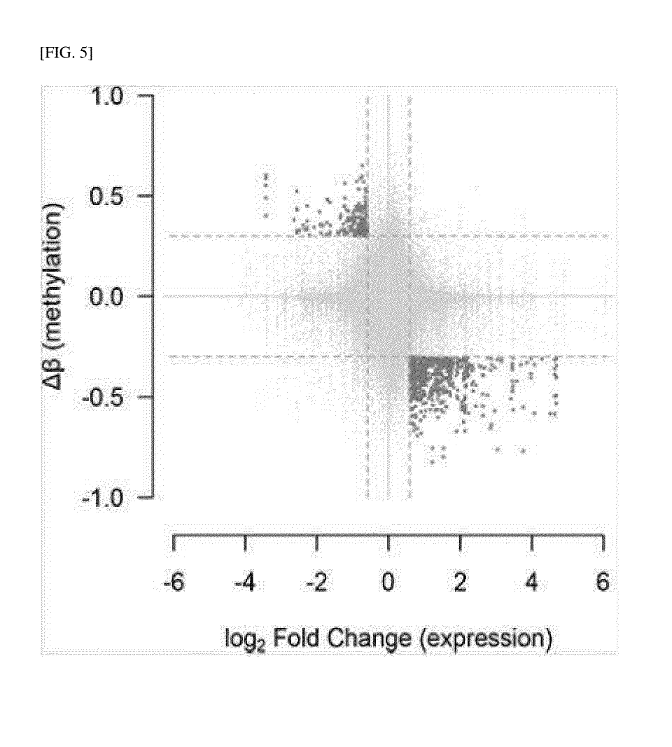

Genes which showed changes in DNA methylation and gene expression in metastatic tumor tissues, compared to the primary ovarian cancer cell line, were selected. Integration analysis of the results was performed to select genes, of which CpG methylation changes were suspected to affect gene expressions (FIG. 5).

From integration of mRNA expression and CpG methylation data, 277 genes of which expressions were increased by hypomethylation at the particular CpG sites in the metastatic group were selected, and 120 genes of which expressions were decreased by hypermethylation at the CpG sites in the metastatic group were selected.

5. Selection of Diagnostic Markers for Ovarian Cancer Metastasis Using Changes in CpG Methylation

The genes, of which CpG methylation changes were suspected to affect gene expressions, were selected by integration analysis, and from the genes, genes which were reported to have functions related to cancer metastasis were secondly selected. Changes in the gene expressions were examined by Quantitative real-time PCR, so as to select metastasis-specific molecular target candidate genes showing significant differences. Further, the primary cell line SK-OV-3 was treated with a demethylating agent, 5-aza-2′-deoxycytidine, and 3 genes (ADAM12, NTN4 and PTGS2) of which expressions were found to be regulated by DNA methylation were finally selected as diagnostic markers for ovarian cancer metastasis using changes in the particular CpG methylation.

TABLE 3

| | | Expression | Expres- | B | |
|---|---|---|---|---|---|
| Gene | GenBank | logFC | sion | differ- | β |
| name | No. | (fold change) | P value | ence | P value |
| ADAM12 | NM_003474 | −1.25 | 0.000011 | 0.33 | 9.98E−07 |
| NTN4 | NM_021229 | −2.08 | 0.000003 | 0.35 | 0.000726 |
| PTGS2 | NM_000963 | −1.07 | 0.000708 | 0.34 | 1.72E−08 |

6. Changes in CpG Methylation of the Selected Genes and Changes in Gene Expression in Tumor Tissues of Ovarian Cancer Metastasis Animal Model

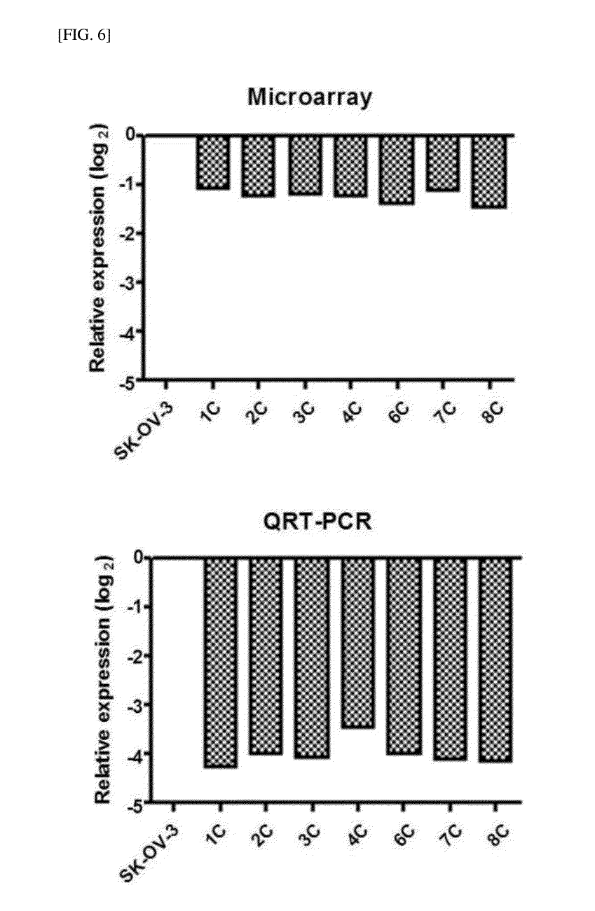

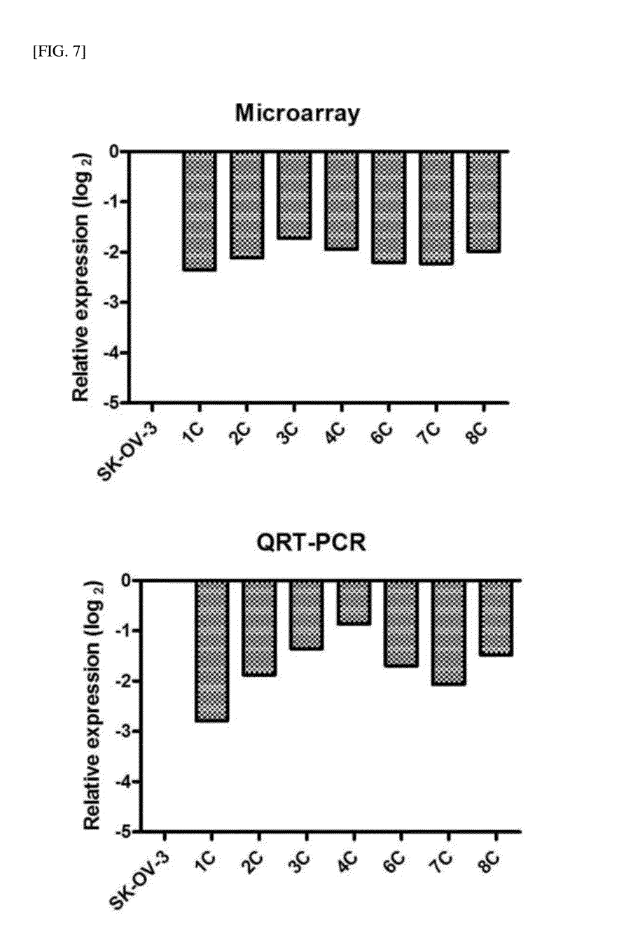

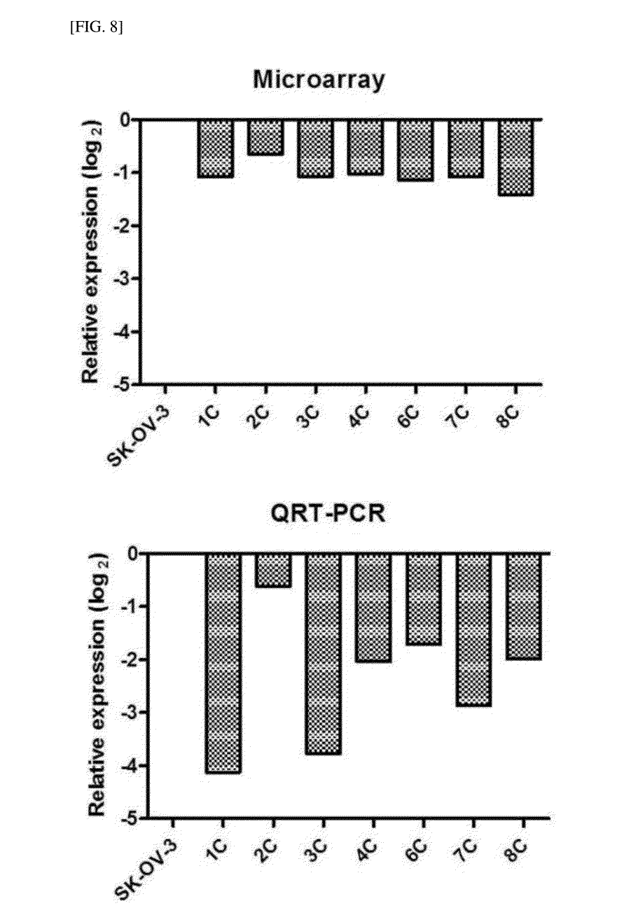

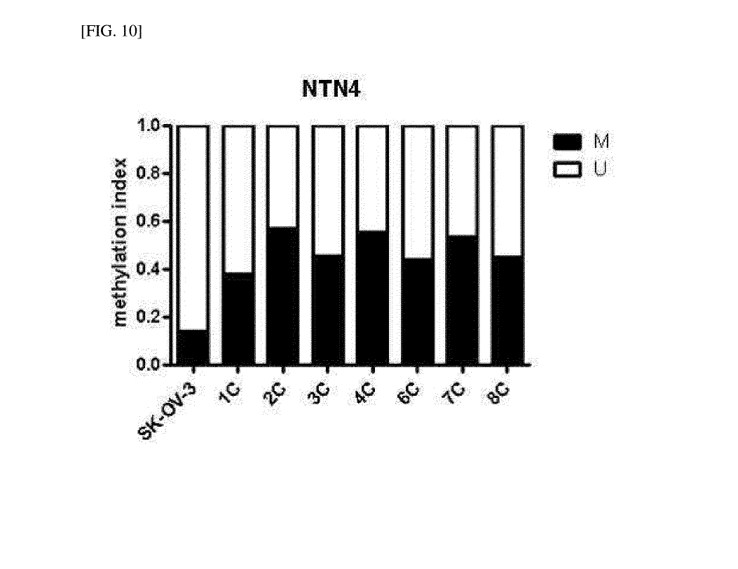

The results of expression microarray showed that expressions of all the three genes (ADAM12, NTN4 and PTGS2) were decreased in the tumor tissues of ovarian cancer metastasis animal model, and the results of qRT-PCR showed similar expression patterns (FIGS. 6 to 10).

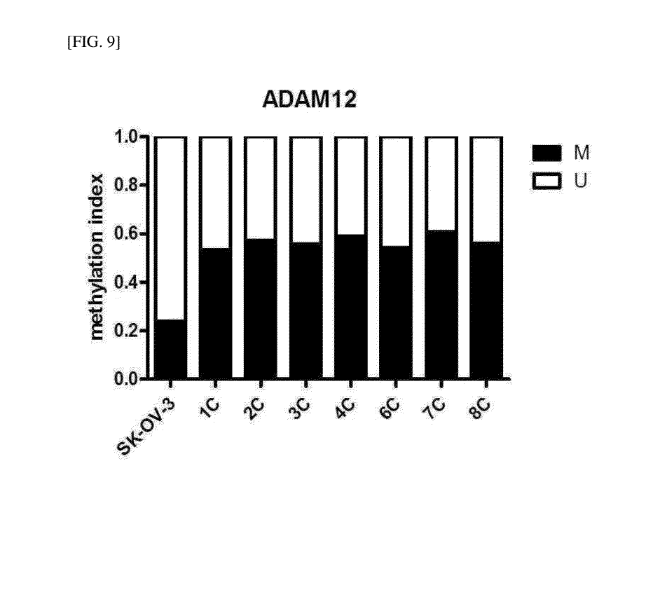

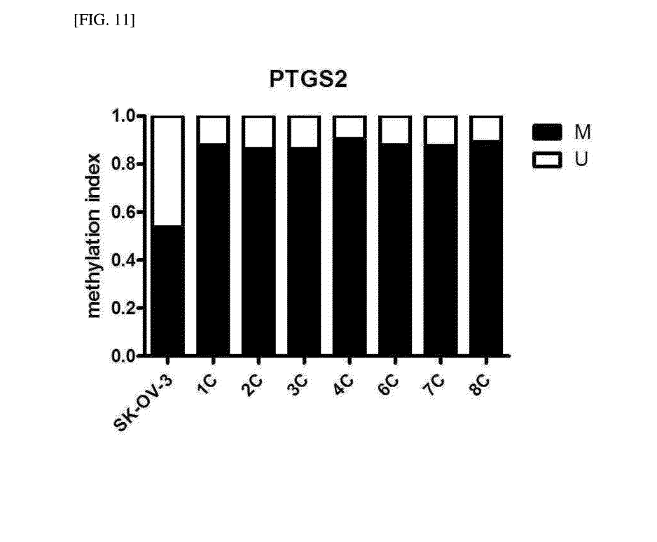

Further, the results of analyzing the DNA methylation microarray showed that DNA methylations at the specific CpG sites of the three genes (ADAM12, NTN4 and PTGS2) were remarkably increased (FIGS. 9 to 11).

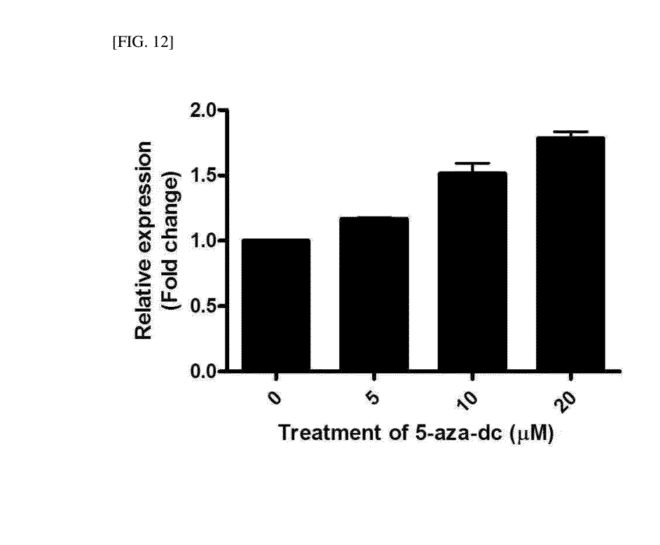

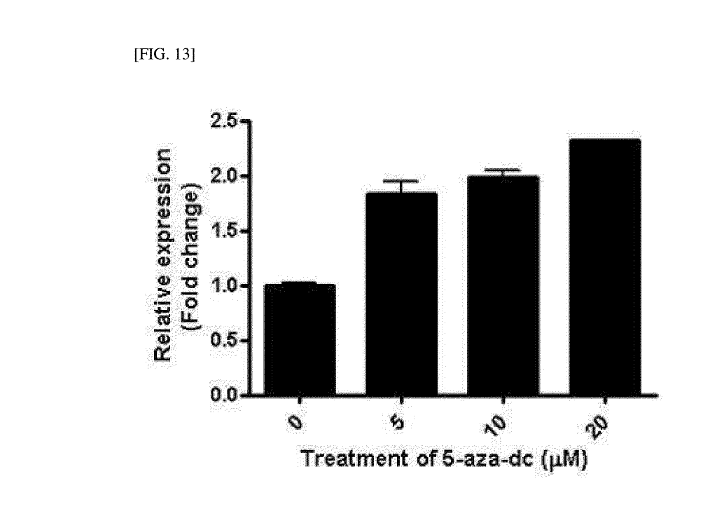

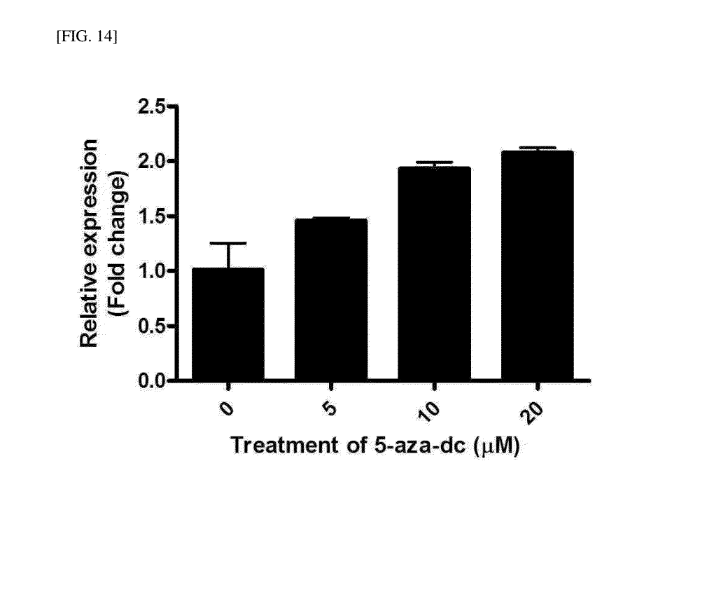

Further, changes in expressions of the three genes (ADAM12, NTN4 and PTGS2) were examined after treatment of the primary cell line SK-OV-3 with the demethylating agent, 5-aza-2′-deoxycytidine for 3 days. As a result, expressions of the above genes were increased with reduced DNA methylation, indicating that the expressions of the above three genes are regulated by DNA methylation (FIGS. 12 to 14).

These experimental results showed that the abrupt decrease in the three genes (ADAM12, NTN4 and PTGS2) in the ovarian cancer metastasis model is regulated by hypermethylation at the specific CpG site of each gene, which is an ovarian cancer metastasis model-specific phenomenon.

1. A composition for diagnosing ovarian cancer metastasis or predicting risk of the metastasis, comprising an agent measuring methylation levels at CpG sites of one or more genes selected from the group consisting of ADAM12 (a disintegrin and metalloproteinase 12), NTN4 (netrin 4) and PTGS2 (prostaglandin-endoperoxide synthase 2).

2. The composition of claim 1, wherein the agent measuring methylation levels at CpG sites of the genes includes a compound modifying an unmethylated cytosine base or a methylation-sensitive restriction enzyme, primers specific to the methylated sequences of the CpG site of ADAM12 gene, and primers specific to the unmethylated sequence thereof.

3. The composition of claim 2, wherein the compound modifying an unmethylated cytosine base is bisulfite or a salt thereof.

4. The composition of claim 2, wherein the methylation sensitive restriction enzyme is SmaI, SacII, EagI, HpaII, MspI, BssHII, BstUI or NotI.

5. The composition of claim 1, wherein the CpG site of the ADAM12 gene includes CpG in the base sequence of SEQ ID NO. 1 (at position from 127779782 to 127779903 of chromosome 10).

6. The composition of claim 1, wherein the CpG site of the NTN4 gene includes CpG in the base sequence of SEQ ID NO. 2 (at position from 96184755 to 96184876 of chromosome 12).

7. The composition of claim 1, wherein the CpG site of the PTGS2 gene includes CpG in the base sequence of SEQ ID NO. 3 (at position from 186650381 to 186650502 of chromosome 1).

8. A kit for diagnosing ovarian cancer metastasis or predicting risk of the metastasis, comprising the composition of claim 1.

9. A method for diagnosing ovarian cancer metastasis or risk of the metastasis, comprising the steps of:
measuring methylation levels at the CpG sites of one or more genes selected from the group consisting of ADAM12 (a disintegrin and metalloproteinase 12), NTN4 (netrin 4) and PTGS2 (prostaglandin-endoperoxide synthase 2) in a biological sample of a subject,
comparing the methylation levels with those of the genes of a control sample, and
determining that the subject has ovarian cancer metastasis or is at the risk of the metastasis, when the methylation levels measured in the sample of the subject are higher than those of the control sample.

10. The method of claim 9, wherein the step (a) is performed by using a compound modifying an unmethylated cytosine base or a methylation sensitive restriction enzyme, primers specific to the methylated sequences of the CpG sites of the genes, and primers specific to the unmethylated sequences thereof.

11. The method of claim 10, wherein the step (a) includes the steps of:
treating genomic DNA obtained from a sample with the compound modifying an unmethylated cytosine base or the methylation sensitive restriction enzyme; and
measuring the methylation level of the treated DNA by one or more methods selected from the group consisting of methylation-specific polymerase chain reaction, real time methylation-specific polymerase chain reaction, PCR using a methylated DNA-specific binding protein, quantitative PCR, pyrosequencing and bisulfite sequencing using primers capable of amplifying the methylated region at the CpG site of the gene.

12. The method of claim 10, wherein the compound modifying an unmethylated cytosine base is bisulfite or a salt thereof.

13. The method of claim 10, wherein the methylation sensitive restriction enzyme is SmaI, SacII, EagI, HpaII, MspI, BssHII, BstUI or NotI.

14. The method of claim 9, wherein the CpG site of the ADAM12 gene includes CpG in the base sequence of SEQ ID NO. 1 (at position from 127779782 to 127779903 of chromosome 10).

15. The method of claim 9, wherein the CpG site of the NTN4 gene includes CpG in the base sequence of SEQ ID NO. 2 (at position from 96184755 to 96184876 of chromosome 12).

16. The method of claim 9, wherein the CpG site of the PTGS2 gene includes CpG in the base sequence of SEQ ID NO. 3 (at position from 186650381 to 186650502 of chromosome 1).
